(19) United States
(12) Patent Application Publication
(10) Pub. No.: US 2026/0363835 A1
(43) Pub. Date: Nov. 19, 2026

(54) SURGICAL INSTRUMENT USED FOR BONE GRAFTING AND METHODS OF USING THE SAME

(71) Applicant: Jonathan Ouellette, Altamonte Springs, FL (US)

(72) Inventor: Jonathan Ouellette, Altamonte Springs, FL (US)

(21) Appl. No.: 19/211,435

(22) Filed: May 19, 2025

(51) Int. Cl. A61C 8/00; A61B 17/16; A61C 19/00

(52) U.S. Cl. CPC A61C 8/0089; A61B 17/1635; A61C 19/005

(57) ABSTRACT

The present invention belongs to the field of surgical instruments, namely for bone grafting. Specifically, the present invention comprises a dental instrument comprising a first tool and a second tool extending therefrom, wherein the first tool is a spatula head and the second tool is a bowl tool, used scooping, transporting, and depositing bone grafting material in dental applications. Methods of using the same are further provided.

### TECHNICAL FIELD

The present invention belongs to the field of surgical instruments, namely for bone grafting. Specifically, the present invention comprises a dental instrument comprising a first tool and a second tool extending therefrom, wherein the first tool is a spatula head and the second tool is a bowl tool, used scooping, transporting, and depositing bone grafting material in dental applications. Methods of using the same are further provided.

### BACKGROUND

Bone grafting is a type of transplantation used to replace missing bone tissue or stimulate the healing of fractures. This surgical procedure is useful for repairing bone fractures that are extremely complex, pose a significant health risk to the patient, or fail to heal properly, which may lead to physical complications.

Bone generally has the ability to regenerate completely but requires a very small fracture space or some sort of scaffold to do so. A bone graft adds bone grafting material to a site requiring bone repair and may be made from bone harvested from a patient's own body, bone harvested from cadavers, or bone material synthetically created and having similar mechanical properties as bone. Most bone grafts are expected to be resorbed and replaced as the natural bone heals over a few months' time.

The most common use of bone grafting is in the application of dental implants to restore the dental jawbone of a missing tooth. Dental bone grafting is a specialized oral surgical procedure that has been developed to reestablish the jawbone infrastructure for support and proper integration into the mouth. This loss can be a result of dental infection of abscess, periodontal disease, trauma, or the natural process of aging.

Most dental instruments utilized in dental bone grafting surgical procedures typically includes a spoon instrument on one side thereof that is used to scoop, remove, and move bone grafting material from a mixing dish to a packing site within the patient's mouth. A packing instrument, either provided separately, or commonly positioned on an opposite side of the spoon instrument is typically used for packing the bone grafting material at the site within the patient's mouth.

In most cases, surgical assistants mix the bone grafting material in the mixing dish. Typically, this is done by blending the bone grafting material with the patient's own blood and/or with saline solution to form a bone grafting material blend. After suitably mixing the bone grafting material to form the bone grafting material blend, typically in the form of a paste, the spoon instrument is used to scoop the same from the mixing bowl to be delivered to the packing site. The surgeon then typically packs the bone grafting material into the packing site using the packing instrument.

As noted, the assistant typically utilizes the spoon for removal of the bone grafting material blend from the mixing bowl and the surgeon typically utilizes the packing instrument for packing the bone grafting material blend at the packing site, often prior to implanting the dental implant. Although the dental instrument typically has a spoon on one side thereof and a packing instrument on the other side thereof, the instrument itself is typically used by two separate individuals: the assistant and the surgeon. This can cause confusion and inefficiencies as both individuals attempt to use the same instrument.

Moreover, the spoon on the one end of the dental instrument typically is used to scoop bone graft material contained within the mixing bowl. However, the spoon is often made of a solid, rigid material, such as metal. The mixing bowl itself is also made of a solid, rigid material, often metal. Thus, when bone grafting material blend is prepared in the mixing bowl, the spoon often has difficulty getting and removing all of the bone grafting material blend from the mixing bowl for use at the packing site. This typically leads to waste of bone grafting material as not all of the bone grafting material blend is typically used and some must be disposed of as waste.

A need, therefore, exists for an improved dental instrument. Specifically, a need exists for an improved dental instrument that improves the use of bone grafting material in dental surgeries, such as dental implant surgeries. More specifically, a need exists for an improved dental instrument that allows the entirety of the bone grafting material to be removed from the mixing bowl thereof for use at a packing site in a dental surgery.

Moreover, a need exists for an improved dental instrument that may be utilized by a singular individual. Specifically, a need exists for an improved dental instrument that is used by an assistant to a dental surgeon in a dental surgery. Moreover, a need exists for an improved dental instrument allowing a single person to scoop bone grafting material from a mixing bowl and ensure that all bone grafting material is used without trading the instrument to another, such as the dental surgeon for packing thereof at the dental surgical site.

### SUMMARY OF THE INVENTION

The present invention belongs to the field of surgical instruments, namely for bone grafting. Specifically, the present invention comprises a dental instrument comprising a first tool and a second tool extending therefrom, wherein the first tool is a spatula head and the second tool is a bowl tool, used scooping, transporting, and depositing bone grafting material in dental applications. Methods of using the same are further provided.

To this end, in an embodiment of the present invention, a dental instrument is provided. The dental instrument comprises: a longitudinal base having a first end and a second end, wherein a spatula head is disposed on the first end of the longitudinal base and a bowl tool is disposed on the second end of the longitudinal base, wherein the spatula head is made from a soft rubbery material.

In an embodiment, the spatula head is connected to the longitudinal base via a neck.

In an embodiment, the neck is thinner than the longitudinal base.

In an embodiment, the neck comprises a tongue, wherein the tongue is embedded within the spatula head.

In an embodiment, the bowl tool is connected to the longitudinal base via a neck.

In an embodiment, the spatula head is made from a silicone material.

In an embodiment, the bowl tool is made from a rigid material.

In an embodiment, the rigid material is a metal.

In an embodiment, the spatula head is connected to the longitudinal base via a first neck and the bowl tool is connected to the longitudinal base via a second neck.

In an embodiment, the longitudinal base has a gripping element over at least a portion of the longitudinal base.

In an alternate embodiment of the present invention, a method of using a dental instrument is provided. The method comprises the steps of: providing a longitudinal base having a first end and a second end, wherein a spatula head is disposed on the first end of the longitudinal base and a bowl tool is disposed on the second end of the longitudinal base, wherein the spatula head is made from a soft rubbery material; scooping material into the bowl tool and transferring the material to a surgical site in a patient's mouth; and scooping additional material into the spatula head and transferring the additional material to the surgical site in a patient's mouth.

In an embodiment, the material comprises a bone grafting material.

In an embodiment, the bone grafting material is mixed with the patient's blood or saline in a mixing bowl and the bowl tool and the spatula head scoops the bone grafting material from the mixing bowl and transfers the bone grafting material to a patient's mouth.

It is, therefore, an advantage and objective of the present invention to provide an improved dental instrument.

Specifically, it is an advantage and objective of the present invention to provide an improved dental instrument that improves the use of bone grafting material in dental surgeries, such as dental implant surgeries.

More specifically, it is an advantage and objective of the present invention to provide an improved dental instrument that allows the entirety of the bone grafting material to be removed from the mixing bowl thereof for use at a packing site in a dental surgery.

Moreover, it is an advantage and objective of the present invention to provide an improved dental instrument that may be utilized by a singular individual.

Specifically, it is an advantage and objective of the present invention to provide an improved dental instrument that is used by an assistant to a dental surgeon in a dental surgery.

Further, it is an advantage and objective of the present invention to provide an improved dental instrument allowing a single person to scoop bone grafting material from a mixing bowl and ensure that all bone grafting material is used without trading the instrument to another, such as the dental surgeon for packing thereof at the dental surgical site.

Additional features and advantages of the present invention are described in, and will be apparent from, the detailed description of the presently preferred embodiments and from the drawings.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 illustrates a perspective view of a dental instrument in an embodiment of the present invention.

FIGS. 2A and 2B illustrate a dental instrument and a close-up of a first tool on a first end of the dental instrument in an embodiment of the present invention.

FIG. 3 illustrates a side view of a second tool on a second end of a dental instrument in an embodiment of the present invention.

### DETAILED DESCRIPTION OF THE PRESENTLY PREFERRED EMBODIMENTS

The present invention belongs to the field of surgical instruments, namely for bone grafting. Specifically, the present invention comprises a dental instrument comprising a first tool and a second tool extending therefrom, wherein the first tool is a spatula head and the second tool is a bowl tool, used scooping, transporting, and depositing bone grafting material in dental applications. Methods of using the same are further provided.

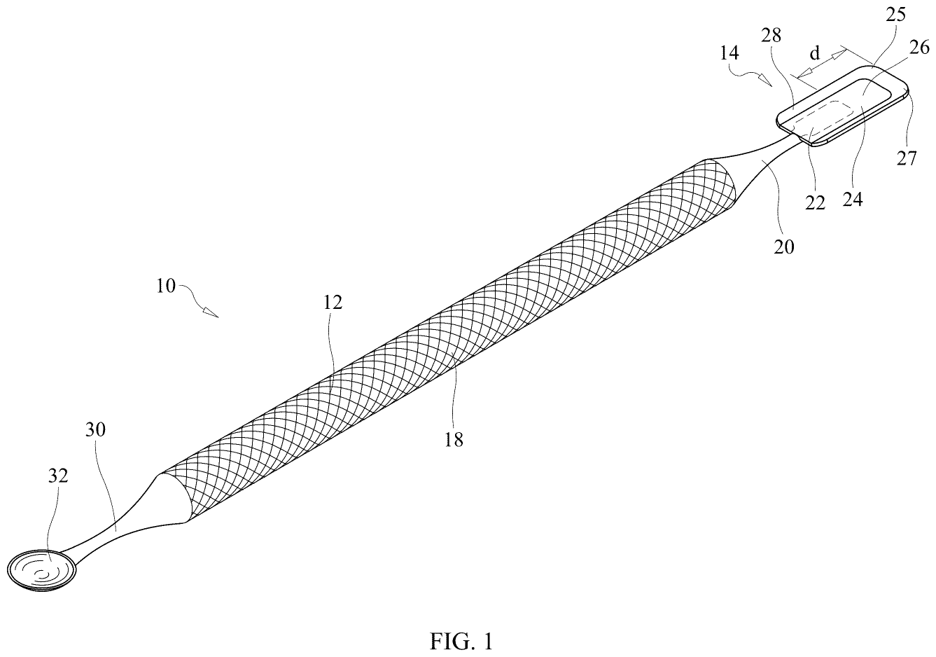

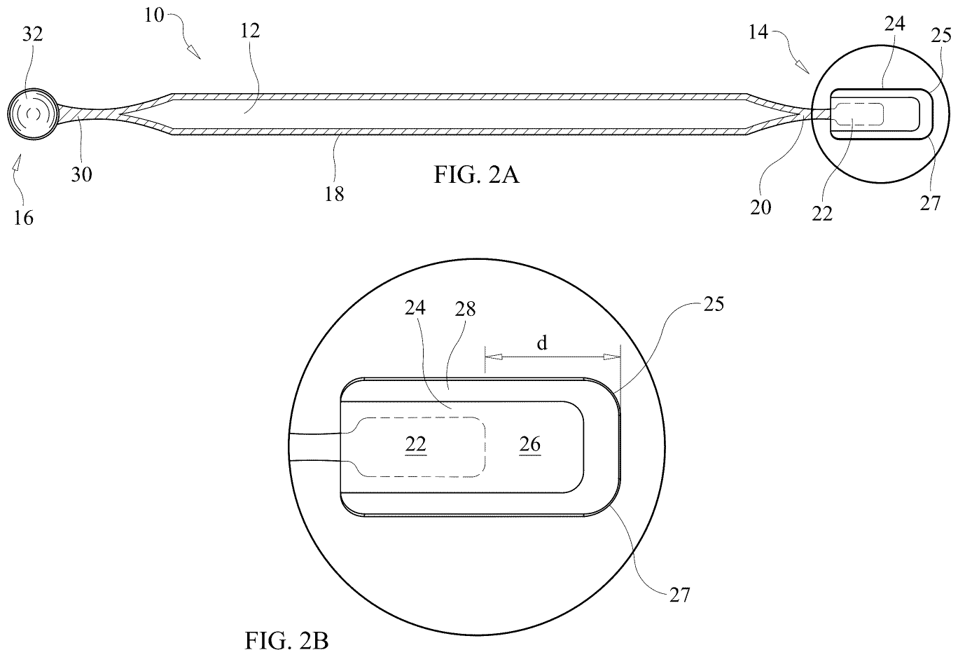

Now referring to the figures, wherein like numerals refer to like parts, FIGS. 1, 2A and 2B illustrate a dental instrument 10 in an embodiment of the present invention. It should be noted that although the present invention describes a dental instrument, it may also include any surgical instrument that may be used for bone grafting, and not just for use in dentistry. Therefore, the use of the term “dental instrument” is meant to include any surgical instrument having the same or similar structure as described herein.

The dental instrument 10 comprises a longitudinal base 12 having a first end 14 and a second end 16 on an opposite side thereof. The base 12 may be sized and shaped to be gripped by a user thereof for using tools disposed on the first end 14 and the second end 16 thereof. For example, the base 12 may be roughly cylindrical and have a gripping element 18 disposed thereon such as, for example, an etched pattern, an elastomeric coating, or any other gripping element apparent to one of ordinary skill in the art.

As shown in FIG. 1, in an exemplary embodiment, the gripping element 18 may be a knurled pattern to provide the texture thereof for gripping the same by a user thereof. In a preferred embodiment, the knurling of the texture may extend over only a portion of the surface of the base 12 and further may be etched at about 90 degrees directions from one another. Moreover, preferably, the knurling pattern may be offset about 45 degrees from the longitudinal axis of the base 12.

On the first end 14 may be a neck 20 extending from the base 12 that may be relatively thinner than the base 12 and a tongue 22 may extend from the neck 20. The tongue 22 may be embedded within a spatula tool head 24 extending from the tongue 22. The spatula tool head 24 may be made from a relatively soft material, such as a rubbery elastomeric material. In a preferred embodiment, the spatula tool head 24 may be made from silicone, which has the advantage of being autoclavable and, therefore, sterilized. Preferably, the spatula tool head 24 may extend a distance -d- from the tongue 22 so that the same in bendable when pressed against a rigid surface.

The spatula tool head 24 may include a flat portion 26 and a rim 28 disposed roughly circumferentially around the flat portion 26. The rim 28 may be raised relative to the flat portion 26 so provide a catchment for material that may be provided on the flat portion 26. For example, the spatula tool head 24 may be used to retrieve bone grafting material from a mixing bowl (not shown). As noted, the spatula tool head 24 may bend when pressed against a side of the mixing bowl to retrieve as much bone grafting material as possible from the mixing bowl. Thus, the spatula head 24 may be utilized to ensure that no bone grafting material is wasted from the mixing bowl and is used within a patient's mouth.

The spatula tool head 24 may have first and second front rounded edges 25, 27 having different radiuses of curvature. For example, the first front rounded edge 25 may have a radius of curvature of about 0.25″ whereas the second front rounded edge 27 may have a radius of curvature of about 0.50″. The different radiuses of curvature between the first and second front rounded edges 25, 27 may aid a user in capturing as much bone grafting material as possible from the mixing bowl.

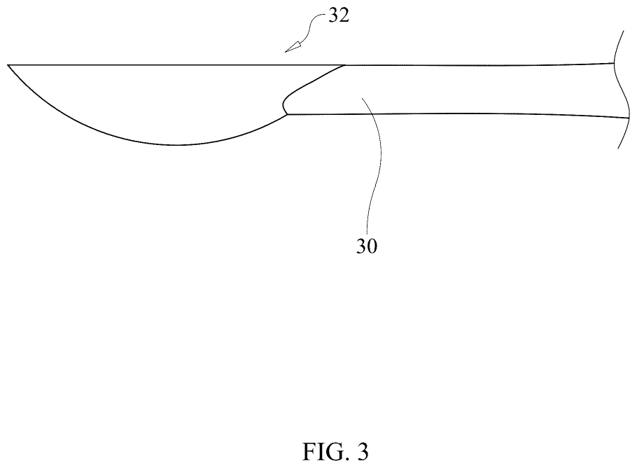

On the second end 16 of the base 12 may be a second neck 30 extending therefrom and a bowl tool 32 on the second neck 30, as further illustrated in FIG. 3, a side view of the bowl tool 32 and the second neck 30. The bowl tool 32 may be roughly semispherical having a size and shape allowing a relatively large amount of material to be captured, moved, and deposited within a patient's mouth during dental surgery. Specifically, the bowl tool 32 may be used to capture most of the bone grafting material that may be within the mixing bowl (not shown) for transfer and depositing in the patient's mouth. The bowl tool 32 may be made from any material; however, in a preferred embodiment, the bowl tool 32 is made of metal and easily sterilized via autoclave.

Thus, the bowl tool 32 may be used to scoop out, transfer and deposit an amount of bone grafting material blended in the mixing bowl. A user may then simply turn the instrument around and capture any remaining bone grafting material within the mixing bowl that may be left behind by using the spatula head 24. Thus, the same user may use both the bowl tool 32 and the spatula head 24 to remove, transfer, and deposit bone grafting material to the surgical site in a patient's mouth.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

I claim:

1. A surgical instrument comprising:
a longitudinal base having a first end and a second end, wherein a spatula head is disposed on the first end of the longitudinal base and a bowl tool is disposed on the second end of the longitudinal base,
wherein the spatula head is made from a soft rubbery material.

2. The surgical instrument of claim 1 wherein the spatula head is connected to the longitudinal base via a neck.

3. The surgical instrument of claim 2 wherein the first neck is thinner than the longitudinal base.

4. The surgical instrument of claim 2 wherein the neck comprises a tongue, wherein the tongue is embedded within the spatula head.

5. The surgical instrument of claim 1 wherein the bowl tool is connected to the longitudinal base via a neck.

6. The surgical instrument of claim 1 wherein the spatula head is made from a silicone material.

7. The surgical instrument of claim 1 wherein the bowl tool is made from a rigid material.

8. The surgical instrument of claim 7 wherein the rigid material is a metal.

9. The surgical instrument of claim 1 wherein the spatula head is connected to the longitudinal base via a first neck and the bowl tool is connected to the longitudinal base via a second neck.

10. The Surgical instrument of claim 1 wherein the longitudinal base has a gripping element over at least a portion of the longitudinal base.

11. A method of using a surgical instrument comprising the steps of:
providing a longitudinal base having a first end and a second end, wherein a spatula head is disposed on the first end of the longitudinal base and a bowl tool is disposed on the second end of the longitudinal base, wherein the spatula head is made from a soft rubbery material;
scooping material into the bowl tool and transferring the material to a surgical site in a patient's mouth; and
scooping additional material into the spatula head and transferring the additional material to the surgical site in a patient's mouth.

12. The method of claim 11 wherein the spatula head is connected to the longitudinal base via a neck.

13. The method of claim 12 wherein the first neck is thinner than the longitudinal base.

14. The method of claim 12 wherein the neck comprises a tongue, wherein the tongue is embedded within the spatula head.

15. The method of claim 11 wherein the bowl tool is connected to the longitudinal base via a neck.

16. The method of claim 11 wherein the spatula head is made from a silicone material.

17. The method of claim 11 wherein the bowl tool is made from a rigid material.

18. The method of claim 11 wherein the spatula head is bendable when used to scoop material therein.

19. The method of claim 11 wherein the material comprises a bone grafting material.

20. The method of claim 11 wherein the bone grafting material is mixed with the patient's blood or saline in a mixing bowl and the bowl tool and the spatula head scoops the bone grafting material from the mixing bowl and transfers the bone grafting material to a patient's mouth.
